(19) United States
(12) Patent Application Publication
(10) Pub. No.: US 2022/0199212 A1
(43) Pub. Date: Jun. 23, 2022

(54) AI Platform System and Method

(71) Applicant: Nuance Communications, Inc., Burlington, MA (US)

(72) Inventors: Jamin L. Wunderink, Cary, NC (US); Rob Smith, Lyndeborough, NH (US)

(21) Appl. No.: 17/558,336

(22) Filed: Dec. 21, 2021

Related U.S. Application Data
(60) Provisional application No. 63/129,301, filed on Dec. 22, 2020.

(51) Int. Cl. G16H 15/00; G16H 30/20

(52) U.S. Cl. CPC G16H 15/00; G16H 30/20

(57) ABSTRACT

A computer-implemented method, computer program product and computing system for defining a test truth set from a master truth set; processing the test truth set using an automated analysis process to generate an automated result set; determining a process efficacy for the automated analysis process based, at least in part, upon the test truth set and the automated result set; and rendering the process efficacy of the automated analysis process.

### RELATED APPLICATION

This application claims the benefit of U.S. Provisional Application No. 63/129,301, filed on 22 Dec. 2020, the entire contents of which is herein incorporated by reference.

### TECHNICAL FIELD

This disclosure relates to platform systems and methods and, more particularly, to platform systems and methods concerning artificial intelligence and machine learning functionality.

### BACKGROUND

Recent advances in the fields of artificial intelligence and machine learning are showing promising outcomes in the analysis of clinical content, examples of which may include medical imagery. Accordingly, processes and algorithms are constantly being developed that may aid in the processing and analysis of such medical imagery. Unfortunately, the efficacy of such processes and algorithms may be less than clear and an interested party may wish to determine how effective a particular process/algorithm is prior to licensing/purchasing the same. Further, the interested party may wish to compare a plurality of processes/algorithms prior to licensing/purchasing the same and/or monitor the continued temporal accuracy of any purchased processes/algorithms.

### SUMMARY OF DISCLOSURE

Concept #3

In one implementation, a computer-implemented method is executed on a computing device and includes: iteratively processing a test truth set using an automated analysis process to generate a plurality of temporarily-spaced automated result sets; iteratively determining a process efficacy for the automated analysis process based, at least in part, upon the test truth set and the plurality of temporarily-spaced automated result sets, thus defining a plurality of temporarily-spaced process efficacies; and determining a long-term efficacy for the automated analysis process based, at least in part, upon the plurality of temporarily-spaced process efficacies.

One or more of the following features may be included. The test truth set may be defined from a master truth set. Defining the test truth set from a master truth set may include: enabling a user to define narrowing criteria for the master truth set; and applying the narrowing criteria to the master truth set to generate the test truth set, wherein the test truth set is a subset of the master truth set. The narrowing criteria may concern one or more of: content type; patient type; and anomaly type. The test truth set may include a plurality of medical images and a plurality of related human-generated reports. The plurality of temporarily-spaced automated result sets may include a plurality of temporarily-spaced machine-generated reports. Iteratively processing a test truth set using an automated analysis process to generate a plurality of temporarily-spaced automated result sets may include: iteratively processing the plurality of medical images using the automated analysis process to generate the plurality of temporarily-spaced machine-generated reports included in the plurality of temporarily-spaced automated result sets, based upon the plurality of medical images. Determining a long-term efficacy for the automated analysis process based, at least in part, upon the plurality of temporarily-spaced process efficacies may include: monitoring the plurality of temporarily-spaced process efficacies to define an efficacy trend for the automated analysis process. Determining a long-term efficacy for the automated analysis process based, at least in part, upon the plurality of temporarily-spaced process efficacies may include: confirming that the efficacy trend is stable/trending upward. Determining a long-term efficacy for the automated analysis process based, at least in part, upon the plurality of temporarily-spaced process efficacies may include: confirming that the efficacy trend is not trending downward.

In another implementation, a computer program product resides on a computer readable medium and has a plurality of instructions stored on it. When executed by a processor, the instructions cause the processor to perform operations including: iteratively processing a test truth set using an automated analysis process to generate a plurality of temporarily-spaced automated result sets; iteratively determining a process efficacy for the automated analysis process based, at least in part, upon the test truth set and the plurality of temporarily-spaced automated result sets, thus defining a plurality of temporarily-spaced process efficacies; and determining a long-term efficacy for the automated analysis process based, at least in part, upon the plurality of temporarily-spaced process efficacies.

One or more of the following features may be included. The test truth set may be defined from a master truth set. Defining the test truth set from a master truth set may include: enabling a user to define narrowing criteria for the master truth set; and applying the narrowing criteria to the master truth set to generate the test truth set, wherein the test truth set is a subset of the master truth set. The narrowing criteria may concern one or more of: content type; patient type; and anomaly type. The test truth set may include a plurality of medical images and a plurality of related human-generated reports. The plurality of temporarily-spaced automated result sets may include a plurality of temporarily-spaced machine-generated reports. Iteratively processing a test truth set using an automated analysis process to generate a plurality of temporarily-spaced automated result sets may include: iteratively processing the plurality of medical images using the automated analysis process to generate the plurality of temporarily-spaced machine-generated reports included in the plurality of temporarily-spaced automated result sets, based upon the plurality of medical images. Determining a long-term efficacy for the automated analysis process based, at least in part, upon the plurality of temporarily-spaced process efficacies may include: monitoring the plurality of temporarily-spaced process efficacies to define an efficacy trend for the automated analysis process. Determining a long-term efficacy for the automated analysis process based, at least in part, upon the plurality of temporarily-spaced process efficacies may include: confirming that the efficacy trend is stable/trending upward. Determining a long-term efficacy for the automated analysis process based, at least in part, upon the plurality of temporarily-spaced process efficacies may include: confirming that the efficacy trend is not trending downward.

In another implementation, a computing system includes a processor and a memory system configured to perform operations including: iteratively processing a test truth set using an automated analysis process to generate a plurality of temporarily-spaced automated result sets; iteratively determining a process efficacy for the automated analysis process based, at least in part, upon the test truth set and the plurality of temporarily-spaced automated result sets, thus defining a plurality of temporarily-spaced process efficacies; and determining a long-term efficacy for the automated analysis process based, at least in part, upon the plurality of temporarily-spaced process efficacies.

One or more of the following features may be included. The test truth set may be defined from a master truth set. Defining the test truth set from a master truth set may include: enabling a user to define narrowing criteria for the master truth set; and applying the narrowing criteria to the master truth set to generate the test truth set, wherein the test truth set is a subset of the master truth set. The narrowing criteria may concern one or more of: content type; patient type; and anomaly type. The test truth set may include a plurality of medical images and a plurality of related human-generated reports. The plurality of temporarily-spaced automated result sets may include a plurality of temporarily-spaced machine-generated reports. Iteratively processing a test truth set using an automated analysis process to generate a plurality of temporarily-spaced automated result sets may include: iteratively processing the plurality of medical images using the automated analysis process to generate the plurality of temporarily-spaced machine-generated reports included in the plurality of temporarily-spaced automated result sets, based upon the plurality of medical images. Determining a long-term efficacy for the automated analysis process based, at least in part, upon the plurality of temporarily-spaced process efficacies may include: monitoring the plurality of temporarily-spaced process efficacies to define an efficacy trend for the automated analysis process. Determining a long-term efficacy for the automated analysis process based, at least in part, upon the plurality of temporarily-spaced process efficacies may include: confirming that the efficacy trend is stable/trending upward. Determining a long-term efficacy for the automated analysis process based, at least in part, upon the plurality of temporarily-spaced process efficacies may include: confirming that the efficacy trend is not trending downward.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a distributed computing network including a computing device that executes an online platform process according to an embodiment of the present disclosure;

FIG. 2 is a flowchart of one implementation of the online platform process of FIG. 1 according to an embodiment of the present disclosure;

FIG. 3 is a diagrammatic view of a user interface rendered by the online platform process of FIG. 1 according to an embodiment of the present disclosure;

FIG. 4 is a flowchart of another implementation of the online platform process of FIG. 1 according to an embodiment of the present disclosure;

FIG. 5 is a diagrammatic view of another user interface rendered by the online platform process of FIG. 1 according to an embodiment of the present disclosure;

FIG. 6 is a flowchart of another implementation of the online platform process of FIG. 1 according to an embodiment of the present disclosure; and

FIG. 7 is a diagrammatic view of another user interface rendered by the online platform process of FIG. 1 according to an embodiment of the present disclosure.

Like reference symbols in the various drawings indicate like elements.

### DETAILED DESCRIPTION OF THE PREFERRED EMBODIMENTS

System Overview

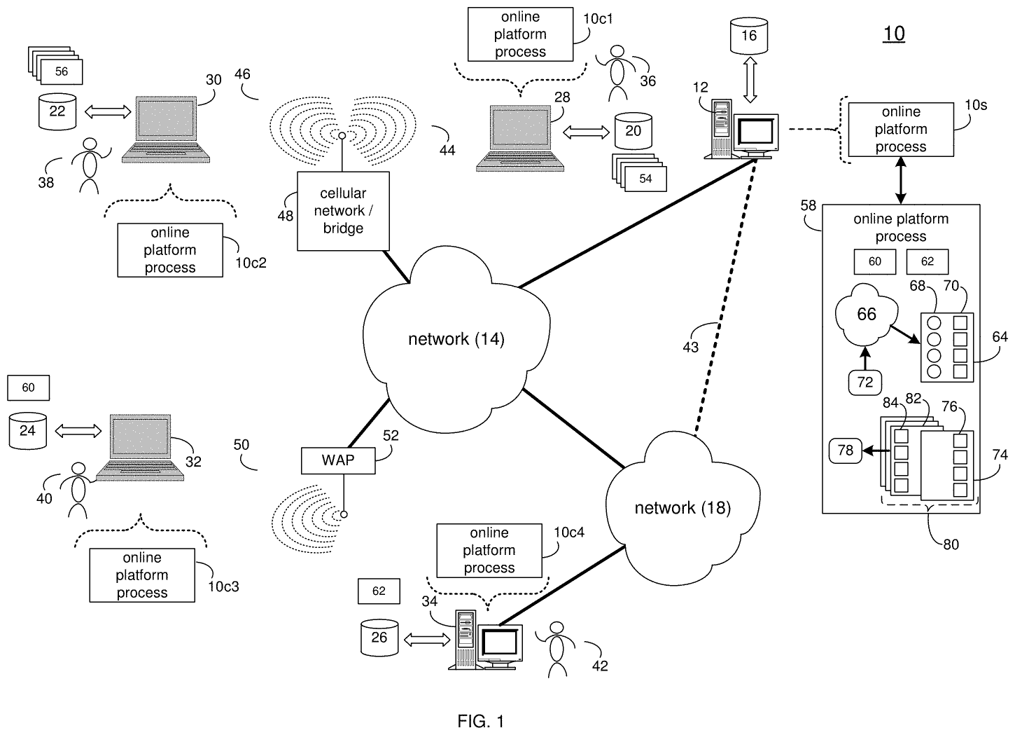

Referring to FIG. 1, there is shown online platform process 10. Online platform process 10 may be implemented as a server-side process, a client-side process, or a hybrid server-side/client-side process. For example, online platform process 10 may be implemented as a purely server-side process via online platform process 10s. Alternatively, online platform process 10 may be implemented as a purely client-side process via one or more of online platform process 10c1, online platform process 10c2, online platform process 10c3, and online platform process 10c4. Alternatively still, online platform process 10 may be implemented as a hybrid server-side/client-side process via online platform process 10s in combination with one or more of online platform process 10c1, online platform process 10c2, online platform process 10c3, and online platform process 10c4. Accordingly, online platform process 10 as used in this disclosure may include any combination of online platform process 10s, online platform process 10c1, online platform process 10c2, online platform process 10c3, and online platform process 10c4. Examples of online platform process 10 may include but are not limited to all or a portion of the PowerShare™ platform and/or the PowerScribe™ platform available from Nuance Communications™ of Burlington, Mass.

Online platform process 10s may be a server application and may reside on and may be executed by computing device 12, which may be connected to network 14 (e.g., the Internet or a local area network). Examples of computing device 12 may include, but are not limited to: a personal computer, a server computer, a series of server computers, a mini computer, a mainframe computer, or a cloud-based computing platform.

The instruction sets and subroutines of online platform process 10s, which may be stored on storage device 16 coupled to computing device 12, may be executed by one or more processors (not shown) and one or more memory architectures (not shown) included within computing device 12. Examples of storage device 16 may include but are not limited to: a hard disk drive; a RAID device; a random access memory (RAM); a read-only memory (ROM); and all forms of flash memory storage devices.

Network 14 may be connected to one or more secondary networks (e.g., network 18), examples of which may include but are not limited to: a local area network; a wide area network; or an intranet, for example.

Examples of online platform processes 10c1, 10c2, 10c3, 10c4 may include but are not limited to a web browser, a game console user interface, a mobile device user interface, or a specialized application (e.g., an application running on e.g., the Android™ platform, the iOS™ platform, the Windows™ platform, the Linux™ platform or the UNIX™ platform). The instruction sets and subroutines of online platform processes 10c1, 10c2, 10c3, 10c4, which may be stored on storage devices 20, 22, 24, 26 (respectively) coupled to client electronic devices 28, 30, 32, 34 (respectively), may be executed by one or more processors (not shown) and one or more memory architectures (not shown) incorporated into client electronic devices 28, 30, 32, 34 (respectively). Examples of storage devices 20, 22, 24, 26 may include but are not limited to: hard disk drives; RAID devices; random access memories (RAM); read-only memories (ROM), and all forms of flash memory storage devices.

Examples of client electronic devices 28, 30, 32, 34 may include, but are not limited to, a smartphone (not shown), a personal digital assistant (not shown), a tablet computer (not shown), laptop computers 28, 30, 32, personal computer 34, a notebook computer (not shown), a server computer (not shown), a gaming console (not shown), and a dedicated network device (not shown). Client electronic devices 28, 30, 32, 34 may each execute an operating system, examples of which may include but are not limited to Microsoft Windows™, Android™, iOS™, Linux™, or a custom operating system.

Users 36, 38, 40, 42 may access online platform process 10 directly through network 14 or through secondary network 18. Further, online platform process 10 may be connected to network 14 through secondary network 18, as illustrated with link line 43.

The various client electronic devices (e.g., client electronic devices 28, 30, 32, 34) may be directly or indirectly coupled to network 14 (or network 18). For example, laptop computer 28 and laptop computer 30 are shown wirelessly coupled to network 14 via wireless communication channels 44, 46 (respectively) established between laptop computers 28, 30 (respectively) and cellular network/bridge 48, which is shown directly coupled to network 14. Further, laptop computer 32 is shown wirelessly coupled to network 14 via wireless communication channel 50 established between laptop computer 32 and wireless access point (i.e., WAP) 52, which is shown directly coupled to network 14. Additionally, personal computer 34 is shown directly coupled to network 18 via a hardwired network connection.

WAP 52 may be, for example, an IEEE 802.11a, 802.11b, 802.11g, 802.11n, Wi-Fi, and/or Bluetooth device that is capable of establishing wireless communication channel 50 between laptop computer 32 and WAP 52. As is known in the art, IEEE 802.11x specifications may use Ethernet protocol and carrier sense multiple access with collision avoidance (i.e., CSMA/CA) for path sharing. As is known in the art, Bluetooth is a telecommunications industry specification that allows e.g., mobile phones, computers, and personal digital assistants to be interconnected using a short-range wireless connection.

While the following discussion concerns medical imagery, this is for illustrative purposes only and is not intended to be a limitation of this disclosure, as other configurations are possible and are considered to be within the scope of this disclosure. For example, the following discussion may concern any type of clinical content (e.g., DNA sequences, EKG results, EEG results, blood panel results, lab results, etc.) and/or non-medical content.

Assume for the following example that users 36, 38 are medical service providers (e.g., radiologists) in two different medical facilities (e.g., hospitals, labs, diagnostic imaging centers, etc.). Accordingly and during the normal operation of these medical facilities, medical imagery may be generated by e.g., x-ray systems (not shown), MRI systems (not shown), CAT systems (not shown), PET systems (not shown) and ultrasound systems (not shown). For example, assume that user 36 generates medical imagery 54 and user 38 generates medical imagery 56; wherein medical imagery 54 may be stored locally on storage device 20 coupled to laptop computer 28 and medical imagery 56 may be stored locally on storage device 22 coupled to laptop computer 30. When locally storing medical imagery 54, 56, this medical imagery may be stored within e.g., a PACS (i.e., Picture Archiving and Communication System). Additionally/alternatively, the medical imagery (e.g., medical imagery 54, 56) may be stored on a cloud-based storage system (e.g., a cloud-based storage system (not shown) included within online platform 58).

Online platform process 10 may enable online platform 58 that may be configured to allow for the offering of various medical diagnostic services to users (e.g., users 36, 38) of online platform 58. For the following example, assume that user 40 is a medical research facility (e.g., the ABC Center) that performs cancer research. Assume that user 40 produced a process (e.g., automated analysis process 60) that analyzes medical imagery to identify anomalies that may be cancer. Examples of automated analysis process 60 may include but are not limited to an application or an algorithm that may process medical imagery (e.g., medical imagery 54 and medical imagery 56), wherein this application/algorithm may utilize artificial intelligence, machine learning and/or probabilistic modeling when analyzing the medical imagery (e.g., medical imagery 54 and medical imagery 56). Examples of such probabilistic modeling may include but are not limited to discriminative modeling (e.g., a probabilistic model for only the content of interest), generative modeling (e.g., a full probabilistic model of all content), or combinations thereof.

Further assume that user 42 is a medical research corporation (e.g., the XYZ Corporation) that produces applications/algorithms (e.g., automated analysis process 62) that analyze medical imagery to identify anomalies that may be cancer. Examples of automated analysis process 62 may include but are not limited to an application or an algorithm that may process medical imagery (e.g., medical imagery 54 and medical imagery 56), wherein this application/algorithm may utilize artificial intelligence, machine learning algorithms and/or probabilistic modeling when analyzing the medical imagery (e.g., medical imagery 54 and medical imagery 56). Examples of such probabilistic modeling may include but are not limited to discriminative modeling (e.g., a probabilistic model for only the content of interest), generative modeling (e.g., a full probabilistic model of all content), or combinations thereof.

Assume for the following example that user 40 (i.e., the ABC Center) wishes to offer automated analysis process 60 to others (e.g., users 36, 38) so that users 36, 38 may use automated analysis process 60 to process their medical imagery (e.g., medical imagery 54 and medical imagery 56, respectively). Further assume that user 42 (i.e., the XYZ Corporation) wishes to offer automated analysis process 62 to others (e.g., users 36, 38) so that users 36, 38 may use automated analysis process 62 to process their medical imagery (e.g., medical imagery 54 and medical imagery 56, respectively).

Accordingly, online platform process 10 and online platform 58 may allow user 40 (i.e., the ABC Center) and/or user 42 (i.e., the XYZ Corporation) to offer automated analysis process 60 and/or automated analysis process 62 (respectively) for use by e.g., user 36 and/or user 38. Therefore, online platform process 10 and online platform 58 may be configured to allow user 40 (i.e., the ABC Center) and/or user 42 (i.e., the XYZ Corporation) to upload a remote copy of automated analysis process 60 and/or automated analysis process 62 to online platform 58, resulting in automated analysis process 60 and/or automated analysis process 62 (respectively) being available for use via online platform 58.

Generally speaking, online platform process 10 may offer a plurality of computer-based medical diagnostic services (e.g., automated analysis process 60, 62) within the online platform (e.g., online platform 58), wherein online platform process 10 may identify the computer-based medical diagnostic services (e.g., automated analysis process 60, 62) that are available via online platform 58 and users (e.g., user 36, 38) may utilize these computer-based medical diagnostic services (e.g., automated analysis process 60, 62) to process the medical imagery (e.g., medical imagery 54 and medical imagery 56).

Concept #1

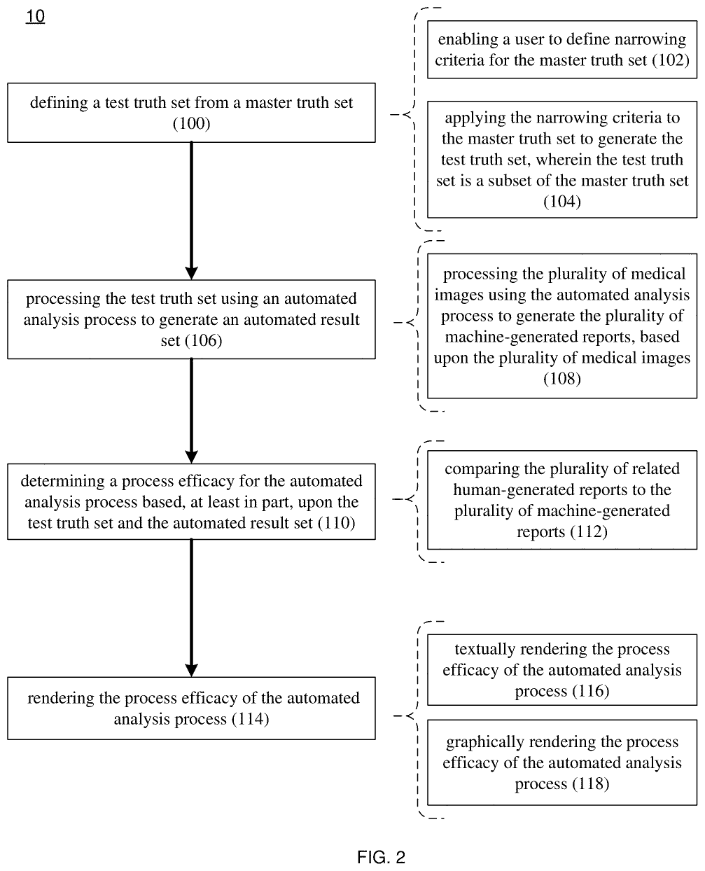

Referring also to FIG. 2, online platform process 10 may define 100 a test truth set (e.g., test truth set 64) from a master truth set (e.g., master truth set 66), wherein the test truth set (e.g., test truth set 64) may include a plurality of medical images (e.g., plurality of medical images 68) and a plurality of related human-generated reports (e.g., plurality of related human-generated reports 70).

As will be discussed below in greater detail, this test truth set (e.g., test truth set 64) may be used by user 36 and/or user 38 to research the available computer-based medical diagnostic services (e.g., automated analysis process 60, 62) to determine which (if any) of these services they would like to e.g., purchase/license/subscribe to.

Generally speaking, master truth set (e.g., master truth set 66) may include/have access to a massive quantity of (in his example) medical images, wherein these medical images may have been reviewed by medical professionals (e.g., radiologists). Medical reports concerning the findings of these medical professionals (e.g., radiologists) with respect to these medical images may be generated, resulting in related human-generated reports. This combination of medical images and related human-generated reports may form the master truth set (e.g., master truth set 66), from which test truth set 64 (which includes plurality of medical images 68 and plurality of related human-generated reports 70) may be defined 100.

For example, plurality of medical images 68 may include an x-ray of the chest of a patient and plurality of related human-generated reports 70 may include a related report that discusses an anomaly within the x-ray that is identified as lung cancer. Additionally, plurality of medical images 68 may include a CT scan of the head of a patient and plurality of related human-generated reports 70 may include a related report that discusses an anomaly within the CT scan that is identified as an intercranial hemorrhage. Further, plurality of medical images 68 may include an MRI scan of the ankle of a patient and plurality of related human-generated reports 70 may include a related report that discusses an anomaly within the MRI scan that is identified as a broken fibula.

While the following discussion concerns the processing of medical imagery, this is for illustrative purposes only and is not intended to be a limitation of this disclosure, as other configurations are possible and are considered to be within the scope of this disclosure. For example, other types of medical information may be processed, such as DNA sequences, EKG results, EEG results, blood panel results, lab results, etc. Additionally, other types of information may be processed that need not be medical in nature. Accordingly and with respect to this disclosure, master truth set 66 may include any type of content for which automated processing may be applicable, such as medical data, financial records, personal records, and identification information.

When defining 100 the test truth set (e.g., test truth set 64) from the master truth set (e.g., master truth set 66), online platform process 10 may enable 102 a user (e.g., user 36 or user 38) to define narrowing criteria (e.g., narrowing criteria 72) for the master truth set (e.g., master truth set 66), wherein the narrowing criteria (e.g., narrowing criteria 72) may concern one or more of: content type; patient type; and anomaly type (as will be discussed below). Further and when defining 100 the test truth set (e.g., test truth set 64) from the master truth set (e.g., master truth set 66), online platform process 10 may apply 104 the narrowing criteria (e.g., narrowing criteria 72) to the master truth set (e.g., master truth set 66) to generate the test truth set (e.g., test truth set 64), wherein the test truth set (e.g., test truth set 64) may be a subset of the master truth set (e.g., master truth set 66).

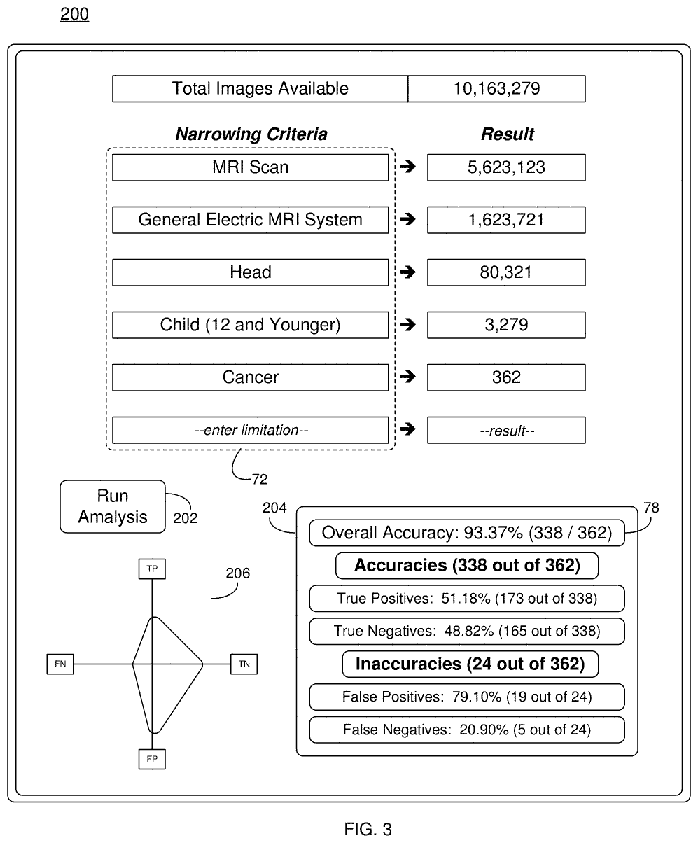

Referring also to FIG. 3, when defining 100 the test truth set (e.g., test truth set 64) from the master truth set (e.g., master truth set 66), online platform process 10 may render user interface 200 that may enable 102 a user (e.g., user 36 or user 38) to define narrowing criteria (e.g., narrowing criteria 72) and may apply 104 the narrowing criteria (e.g., narrowing criteria 72) to the master truth set (e.g., master truth set 66) to generate the test truth set (e.g., test truth set 64). As will be discussed below, the test truth set (e.g., test truth set 64) may be a subset of the master truth set (e.g., master truth set 66).

For example, assume that the master truth set (e.g., master truth set 66) includes 10,163,279 medical images and, therefore, 10,163,279 related human-generated reports. Further assume that e.g., user 36 works at a medical facility that specializes in pediatric neurological issues, wherein user 36 wishes to research the available computer-based medical diagnostic services (e.g., automated analysis process 60, 62) to determine which (if any) of these services is suitable for pediatric neurological issues. As the master truth set (e.g., master truth set 66) includes 10,163,279 medical images/10,163,279 related human-generated reports that may (or may not) concern pediatric neurological issues, user 36 may start to enter narrowing criteria 72 that may whittle away at master truth set 66 to define a test truth set (e.g., test truth set 64) that is related to/pertinent for pediatric neurological issues.

Accordingly, user 36 may enter narrowing criteria 72 that includes:
“MRI Scan”, as the facility in which user 36 works is only interested in the processing of MRI images. This in turn reduces the 10,163,279 medical images/related human-generated reports to 5,623,123 medical images/related human-generated reports.
“General Electric MRI System”, as the facility in which user 36 works uses a General Electric MRI machine. This in turn reduces the 5,623,123 medical images/related human-generated reports to 1,623,721 medical images/related human-generated reports.
“Head”, as the facility in which user 36 works focuses on neurological issues. This in turn reduces the 1,623,721 medical images/related human-generated reports to 80,321 medical images/related human-generated reports.
“Child (12 and Younger)”, as the facility in which user 36 works focuses on pediatric issues. This in turn reduces the 80,321 medical images/related human-generated reports to 3,279 medical images/related human-generated reports.
“Cancer”, as the facility in which user 36 works focuses on cancerous tumors. This in turn reduces the 3,279 medical images/related human-generated reports to 362 medical images/related human-generated reports.

Accordingly and through narrowing criteria 72, a master truth set (e.g., master truth set 66) that includes 10,163,279 medical images/related human-generated reports may be whittled down to a test truth set (e.g., test truth set 64) that is focused on pediatric neurological issues and includes 362 medical images/related human-generated reports (selected from the 10,163,279 medical images/related human-generated reports included within master truth set 66). Accordingly and as stated above, test truth set 64 may be a subset of master truth set 66.

Online platform process 10 may process 106 the test truth set (e.g., test truth set 64) using an automated analysis process (e.g., automated analysis process 60 or automated analysis process 62) to generate an automated result set (e.g., automated result set 74). The automated result set (e.g., automated result set 74) may include a plurality of machine-generated reports (e.g., machine-generated reports 76).

Continuing with the above-stated example, assume that user 36 is interested in automated analysis process 60 offered by the ABC Center (i.e., a cancer research medical facility) . . . but is uncertain as to the manner in which it will perform with respect to pediatric neurological issues. Accordingly and through the use of test truth set 64 (which was curated toward e.g., MRI scans made on General Electric MRI machines that concern pediatric brain cancer), the performance (e.g., accuracy/efficacy) of automated analysis process 60 may be scrutinized.

Accordingly and when processing 106 the test truth set (e.g., test truth set 64) using an automated analysis process (e.g., automated analysis process 60) to generate an automated result set (e.g., automated result set 74), online platform process 10 may process 108 the plurality of medical images (e.g., plurality of medical images 68) using the automated analysis process (e.g., automated analysis process 60) to generate the plurality of machine-generated reports (e.g., plurality of machine-generated reports 76), based upon the plurality of medical images (e.g., plurality of medical images 68).

Generally speaking and if automated analysis process 60 is 100% accurate, the plurality of machine-generated reports (e.g., plurality of machine-generated reports 76) should reach the same conclusion(s) as the plurality of related human-generated reports (e.g., plurality of related human-generated reports 70), as both sets of reports are based upon the same plurality of medical images (e.g., plurality of medical images 68).

Therefore, online platform process 10 may determine 110 a process efficacy (e.g., process efficacy 78) for the automated analysis process (e.g., automated analysis process 60) based, at least in part, upon the test truth set (e.g., test truth set 64) and the automated result set (e.g., automated result set 74).

For example and when determining 110 the process efficacy (e.g., process efficacy 78) for the automated analysis process (e.g., automated analysis process 60) based, at least in part, upon the test truth set (e.g., test truth set 64) and the automated result set (e.g., automated result set 74), online platform process 10 may compare 112 the plurality of related human-generated reports (e.g., plurality of related human-generated reports 70) to the plurality of machine-generated reports (e.g., plurality of machine-generated reports 76). Specifically, the higher the level of correlation between plurality of related human-generated reports 70 and plurality of machine-generated reports 76, the hire the level of efficacy of (in this example) automated analysis process 60.

Accordingly and in this example, once user 36 defines narrowing criteria 72, user 36 may select “Run Analysis” button 202, resulting in online platform process 10 processing 106 test truth set 64 (which includes 362 medical images/related human-generated reports) using automated analysis process 60 to generate automated result set 74; thus allowing online platform process 10 to determine 110 process efficacy 78 for automated analysis process 60 based, at least in part, upon test truth set 64 and automated result set 74.

Online platform process 10 may render 114 the process efficacy (e.g., process efficacy 78) of the automated analysis process (e.g., automated analysis process 60).

For example and when rendering 114 the process efficacy (e.g., process efficacy 78) of the automated analysis process (e.g., automated analysis process 60), online platform process 10 may textually render 116 the process efficacy (e.g., process efficacy 78) of the automated analysis process (e.g., automated analysis process 60).

In this particular illustrative example and as shown within result window 204 of user interface 200, efficacy 78 is shown to be 93.37%, in that automated analysis process 60 produced 338 machine-generated reports (out of a total of 362 machine-generated reports) that reached the same conclusion(s) as the corresponding human-generated report within truth set 64.

Concerning the 338 accurate results, 173 of the 338 results (i.e., 51.18%) were deemed to be “True Positives”, wherein an anomaly was detected and was properly identified as being malignant; and 165 of the 338 results (i.e., 48.82%) were deemed to be “True Negatives”, wherein an anomaly was detected and was properly identified as being benign.

Concerning the 24 inaccurate results, 19 of the 24 results (i.e., 79.10%) were deemed to be “False Positives”, wherein an anomaly was detected and was improperly identified as being malignant; and 5 of the 24 results (i.e., 20.90%) were deemed to be “False Negatives”, wherein an anomaly was detected and was improperly identified as being benign.

Additionally/alternatively and when rendering 114 the process efficacy (e.g., process efficacy 78) of the automated analysis process (e.g., automated analysis process 60), online platform process 10 may graphically render 118 the process efficacy (e.g., process efficacy 78) of the automated analysis process (e.g., automated analysis process 60). For example, online platform process 10 may graphically render 118 a multi-axis spider plot (e.g., graph 206) within user interface 200 that visually identifies True Positives, True Negatives, False Positives, and False Negatives with respect to process efficacy 78 of automated analysis process 60.

Concept #2

As will be discussed below in greater detail, online platform process 10 may allow a user (e.g., user 36) to compare the performance of multiple computer-based medical diagnostic services (e.g., automated analysis process 60, 62) in order to enable the user to determine which (if any) of these services they would like to e.g., purchase/license/subscribe to.

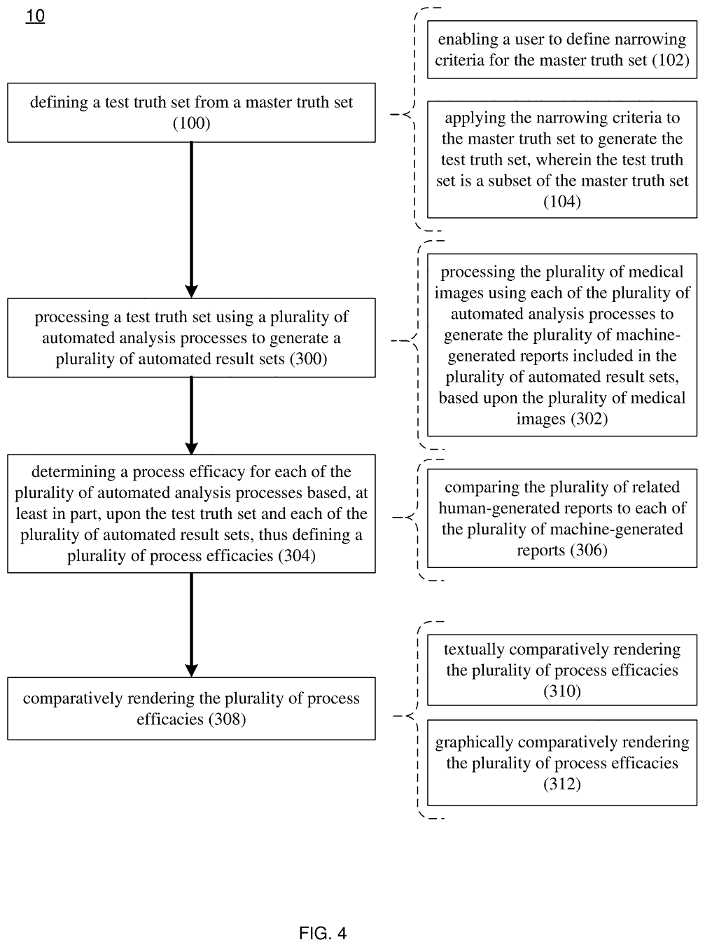

As discussed above and referring also to FIG. 4, online platform process 10 may define 100 the test truth set (e.g., test truth set 64) from a master truth set (e.g., master truth set 66), wherein the test truth set (e.g., test truth set 64) may include a plurality of medical images (e.g., plurality of medical images 68) and a plurality of related human-generated reports (e.g., plurality of related human-generated reports 70).

As also discussed above, when defining 100 the test truth set (e.g., test truth set 64) from a master truth set (e.g., master truth set 66), online platform process 10 may enable 102 a user (e.g., user 36 or user 38) to define narrowing criteria (e.g., narrowing criteria 72) for the master truth set (e.g., master truth set 66) and apply 104 the narrowing criteria (e.g., narrowing criteria 72) to the master truth set (e.g., master truth set 66) to generate the test truth set (e.g., test truth set 64), wherein the test truth set (e.g., test truth set 64) is a subset of the master truth set (e.g., master truth set 66). The narrowing criteria (e.g., narrowing criteria 72) may concern one or more of: content type; patient type; and anomaly type.

Suppose for this example that the user (e.g., user 36) is interested in both computer-based medical diagnostic services (e.g., automated analysis process 60, 62) but does not know which (if any) of these services to e.g., purchase/license/subscribe to.

Accordingly, online platform process 10 may process 300 the test truth set (e.g., test truth set 64) using a plurality of automated analysis processes (e.g., automated analysis processes 60, 62) to generate a plurality of automated result sets (e.g., plurality of automated result sets 80), wherein the plurality of automated result sets (e.g., plurality of automated result sets 80) may each include a plurality of machine-generated reports (an example of which is machine-generated reports 76 included within automated result set 74).

When processing 300 a test truth set (e.g., test truth set 64) using a plurality of automated analysis processes (e.g., automated analysis processes 60, 62) to generate a plurality of automated result sets (e.g., automated result sets 78), online platform process 10 may process 302 the plurality of medical images (e.g., plurality of medical images 68) using each of the plurality of automated analysis processes (e.g., automated analysis processes 60, 62) to generate the plurality of machine-generated reports (an example of which is machine-generated reports 76 included within automated result set 74) included in the plurality of automated result sets (e.g., automated result sets 80), based upon the plurality of medical images (e.g., plurality of medical images 68).

In this situation, being two automated analysis processes (e.g., automated analysis processes 60, 62) are being evaluated by user 36, the plurality of automated result sets (e.g., plurality of automated result sets 80) may include two automated result sets, namely: automated result set 74 which includes machine-generated reports 76 that were generated using automated analysis process 60; and automated result set 82 which includes machine-generated reports 84 that were generated using automated analysis processes 62.

In a similar fashion as described above, online platform process 10 may determine 304 a process efficacy (e.g., process efficacy 78) for each of the plurality of automated analysis processes (e.g., automated analysis processes 60, 62) based, at least in part, upon the test truth set (e.g., test truth set 64) and each of the plurality of automated result sets (e.g., automated result set 74 for automated analysis process 60 and automated result set 82 for automated analysis processes 62), thus defining a plurality of process efficacies (as will be discussed below).

When determining 304 a process efficacy (e.g., process efficacy 78) for each of the plurality of automated analysis processes (e.g., automated analysis processes 60, 62) based, at least in part, upon the test truth set (e.g., test truth set 64) and each of the plurality of automated result sets (e.g., automated result set 74 for automated analysis process 60 and automated result set 82 for automated analysis processes 62), thus defining a plurality of process efficacies (as will be discussed below), online platform process 10 may compare 306 the plurality of related human-generated reports (e.g., plurality of related human-generated reports 70) to each of the plurality of machine-generated reports.

Specifically and when determining 304 a process efficacy for automated analysis process 60, online platform process 10 may compare 306 plurality of related human-generated reports 70 to machine-generated reports 76 that are included within automated result set 74 that was generated using automated analysis process 60. Further and when determining 304 a process efficacy for automated analysis process 62, online platform process 10 may compare 306 plurality of related human-generated reports 70 to machine-generated reports 84 that are included within automated result set 82 that was generated using automated analysis process 62.

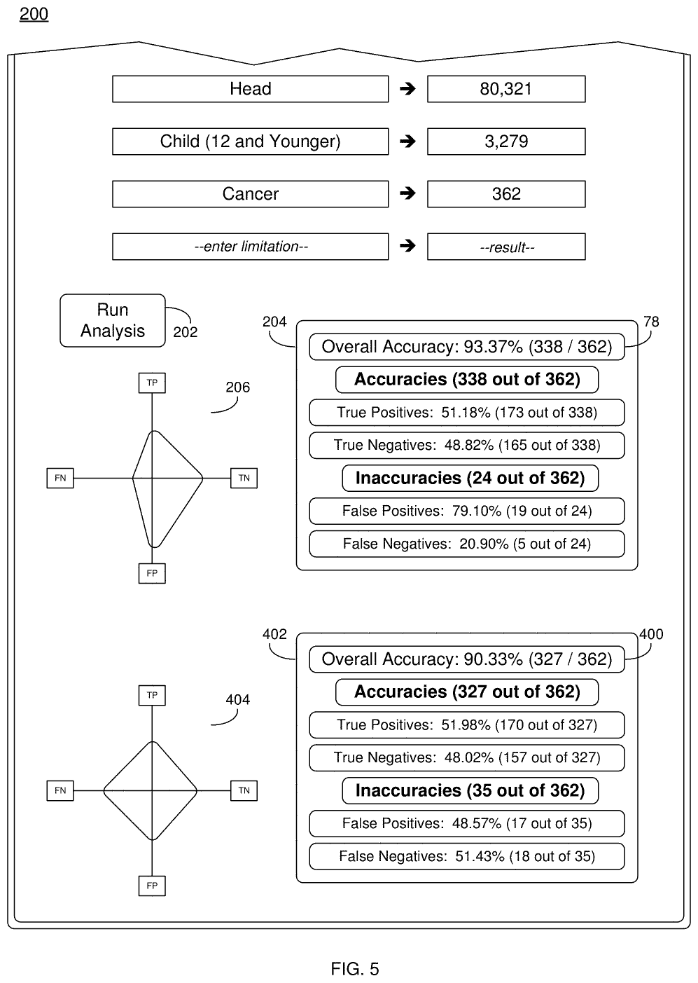

Referring also to FIG. 5, assume that user 36 enters the same narrowing criteria 72, namely:
“MRI Scan”, as the facility in which user 36 works is only interested in the processing of MRI images. This in turn reduces the 10,163,279 medical images/related human-generated reports to 5,623,123 medical images/related human-generated reports.
“General Electric MRI System”, as the facility in which user 36 works uses a General Electric MRI machine. This in turn reduces the 5,623,123 medical images/related human-generated reports to 1,623,721 medical images/related human-generated reports.
“Head”, as the facility in which user 36 works focuses on neurological issues. This in turn reduces the 1,623,721 medical images/related human-generated reports to 80,321 medical images/related human-generated reports.
“Child (12 and Younger)”, as the facility in which user 36 works focuses on pediatric issues. This in turn reduces the 80,321 medical images/related human-generated reports to 3,279 medical images/related human-generated reports.
“Cancer”, as the facility in which user 36 works focuses on cancerous tumors. This in turn reduces the 3,279 medical images/related human-generated reports to 362 medical images/related human-generated reports.

As discussed above and through narrowing criteria 72, a master truth set (e.g., master truth set 66) that includes 10,163,279 medical images/related human-generated reports may be whittled down to a test truth set (e.g., test truth set 64) that is focused on pediatric neurological issues and includes 362 medical images/related human-generated reports (selected from the 10,163,279 medical images/related human-generated reports included within master truth set 66).

Once user 36 defines narrowing criteria 72, user 36 may select “Run Analysis” button 202, resulting in online platform process 10 processing 300 test truth set 64 (which includes 362 medical images/related human-generated reports) using automated analysis process 60 and automated analysis process 62 to generate automated result set 74 that was generated using automated analysis process 60 and automated result set 82 that was generated using automated analysis process 62, thus allowing online platform process 10 to determine 304 a process efficacy (e.g., process efficacies 78, 400) for each of the plurality of automated analysis processes (e.g., automated analysis processes 60, 62) based, at least in part, upon the test truth set (e.g., test truth set 64) and each of the plurality of automated result sets (e.g., automated result set 74 for automated analysis process 60 and automated result set 82 for automated analysis processes 62), thus defining a plurality of process efficacies (e.g., plurality of process efficiencies 78, 400).

Online platform process 10 may comparatively render 308 the plurality of process efficacies (e.g., plurality of process efficiencies 78, 400). For example and when comparatively rendering 308 the plurality of process efficacies (e.g., process efficacies 78, 400), online platform process 10 may textually comparatively render 310 the plurality of process efficacies (e.g., plurality of process efficiencies 78, 400).

In this particular illustrative example and as shown within result window 204 of user interface 200 and with respect to automated analysis process 60, efficacy 78 is shown to be 93.37%, in that automated analysis process 60 produced 338 machine-generated reports (out of a total of 362 machine-generated reports) that reached the same conclusion(s) as the corresponding human-generated report within truth set 64.

Concerning the 338 accurate results, 173 of the 338 results (i.e., 51.18%) were deemed to be “True Positives”, wherein an anomaly was detected and was properly identified as being malignant; and 165 of the 338 results (i.e., 48.82%) were deemed to be “True Negatives”, wherein an anomaly was detected and was properly identified as being benign.

Concerning the 24 inaccurate results, 19 of the 24 results (i.e., 79.10%) were deemed to be “False Positives”, wherein an anomaly was detected and was improperly identified as being malignant; and 5 of the 24 results (i.e., 20.90%) were deemed to be “False Negatives”, wherein an anomaly was detected and was improperly identified as being benign.

In this particular illustrative example and as shown within result window 402 of user interface 200 and with respect to automated analysis process 62, efficacy 400 is shown to be 90.33%, in that automated analysis process 60 produced 327 machine-generated reports (out of a total of 362 machine-generated reports) that reached the same conclusion(s) as the corresponding human-generated report within truth set 64.

Concerning the 327 accurate results, 170 of the 327 results (i.e., 51.98%) were deemed to be “True Positives”, wherein an anomaly was detected and was properly identified as being malignant; and 157 of the 327 results (i.e., 48.02%) were deemed to be “True Negatives”, wherein an anomaly was detected and was properly identified as being benign.

Concerning the 35 inaccurate results, 17 of the 35 results (i.e., 48.57%) were deemed to be “False Positives”, wherein an anomaly was detected and was improperly identified as being malignant; and 18 of the 35 results (i.e., 51.43%) were deemed to be “False Negatives”, wherein an anomaly was detected and was improperly identified as being benign.

When comparatively rendering 308 the plurality of process efficacies, online platform process 10 may graphically comparatively render 312 the plurality of process efficacies (e.g., plurality of process efficiencies 78, 400). For example, online platform process 10 may graphically comparatively render 312 a multi-axis spider plot (e.g., graph 206) within user interface 200 that visually identifies True Positives, True Negatives, False Positives, and False Negatives with respect to process efficacy 78 of automated analysis process 60. Further, online platform process 10 may graphically comparatively render 312 a multi-axis spider plot (e.g., graph 404) within user interface 200 that visually identifies True Positives, True Negatives, False Positives, and False Negatives with respect to process efficacy 400 of automated analysis process 62.

Concept #3

As will be discussed below in greater detail, online platform process 10 may allow a user (e.g., user 36) to monitor the performance of a computer-based medical diagnostic service (e.g., automated analysis process 60, 62) over time to enable the user to determine how the efficacy of the computer-based medical diagnostic service (e.g., automated analysis process 60, 62) changes over time (if at all).

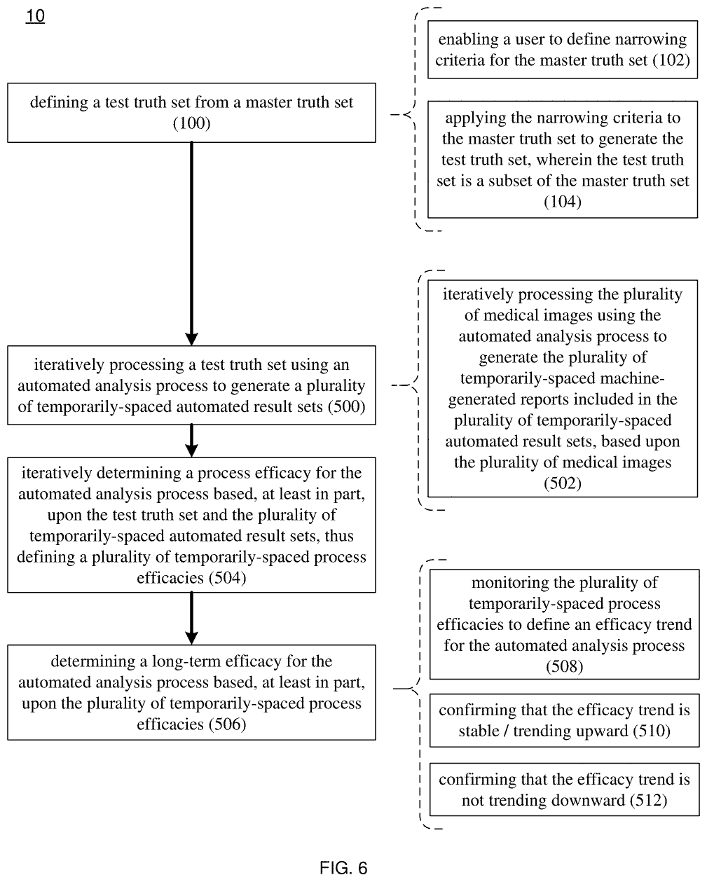

As discussed above and referring also to FIG. 6, online platform process 10 may define 100 the test truth set (e.g., test truth set 64) from a master truth set (e.g., master truth set 66). wherein the test truth set (e.g., test truth set 64) may include a plurality of medical images (e.g., plurality of medical images 68) and a plurality of related human-generated reports (e.g., plurality of related human-generated reports 70).

As also discussed above, when defining 100 the test truth set (e.g., test truth set 64) from a master truth set (e.g., master truth set 66), online platform process 10 may enable 102 a user (e.g., user 36 or user 38) to define narrowing criteria (e.g., narrowing criteria 72) for the master truth set (e.g., master truth set 66); and apply 102 the narrowing criteria (e.g., narrowing criteria 72) to the master truth set (e.g., master truth set 66) to generate the test truth set (e.g., test truth set 64), wherein the test truth set (e.g., test truth set 64) is a subset of the master truth set (e.g., master truth set 66). The narrowing criteria (e.g., narrowing criteria 72) may concern one or more of: content type; patient type; and anomaly type.

Suppose for this example that the user (e.g., user 36) purchased/licensed/subscribed to automated analysis process 60 and would like to know if the efficacy of automated analysis process 60 “ages” well. As discussed above and with respect to automated analysis process 60, efficacy 78 was initially determined to be 93.37%. However and as is known in the art, computer-based medical diagnostic services are continuously learning/evolving based upon additional data that is used to train the computer-based medical diagnostic services. Accordingly, it is foreseeable that the efficacy of a computer-based medical diagnostic service may degrade if bad data is used to train the computer-based medical diagnostic service.

Accordingly and in order to monitor such long-term efficacy and the evolvement of the same, online platform process 10 may iteratively process 500 a test truth set (e.g., test truth set 64) using an automated analysis process (e.g., automated analysis process 60) to generate a plurality of temporarily-spaced automated result sets (e.g., plurality of automated result sets 80).

When iteratively processing 500 a test truth set (e.g., test truth set 64) using an automated analysis process (e.g., automated analysis process 60) to generate a plurality of temporarily-spaced automated result sets (e.g., plurality of automated result sets 80), online platform process 10 may iteratively process 502 the plurality of medical images (e.g., plurality of medical images 68) using the automated analysis process (e.g., automated analysis process 60) to generate the plurality of temporarily-spaced machine-generated reports included in the plurality of temporarily-spaced automated result sets (e.g., plurality of automated result sets 80), based upon the plurality of medical images (e.g., plurality of medical images 68).

As discussed above, each of the automated result sets (e.g., automated result set 74) includes a plurality of machine-generated reports (e.g., machine-generated reports 76). Accordingly, the plurality of temporarily-spaced automated result sets (e.g., plurality of automated result sets 80) may each include a plurality of temporarily-spaced machine-generated reports.

Online platform process 10 may iteratively determine 504 a process efficacy (e.g., process efficacy 78) for the automated analysis process (e.g., automated analysis process 60) based, at least in part, upon the test truth set (e.g., test truth set 64) and the plurality of temporarily-spaced automated result sets (e.g., plurality of automated result sets 80), thus defining a plurality of temporarily-spaced process efficacies (as will be discussed below).

Accordingly, online platform process 10 may iteratively process 502 the plurality of medical images (e.g., plurality of medical images 68) using the automated analysis process (e.g., automated analysis process 60) at a period of e.g., once every three months, thus generating one temporarily-spaced automated result set every three months. Importantly, the same test truth set (e.g., test truth set 64) is used by automated analysis process 60 to generate each of these temporarily-spaced automated result sets (e.g., plurality of automated result sets 80).

Online platform process 10 may then iteratively determine 504 a process efficacy (e.g., process efficacy 78) for the automated analysis process (e.g., automated analysis process 60) based, at least in part, upon the test truth set (e.g., test truth set 64) and each of these temporarily-spaced automated result sets (e.g., plurality of automated result sets 80), thus defining (in this example) a series of temporarily-spaced process efficacies that define the manner in which these efficacies changes with respect to time (i.e., in three month intervals in this example).

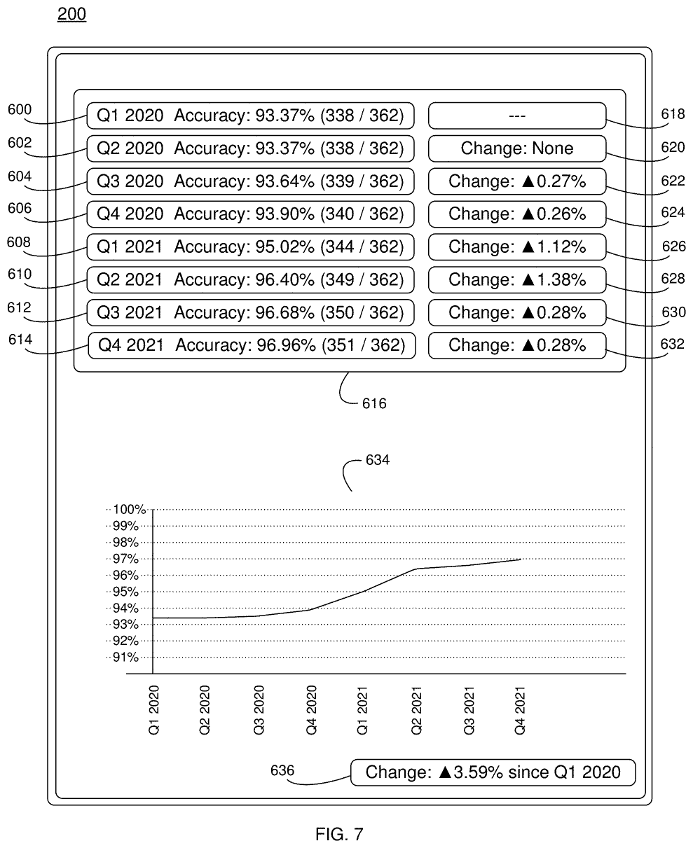

For this particular example and referring also to FIG. 7, online platform process 10 may iteratively determine 504 a process efficacy for automated analysis process 60 once every three months (from Q1 2020 through Q4 2021), resulting in the generation of eight temporarily-spaced process efficacies (namely temporarily-spaced process efficacies 600 (for Q1 2020), 602 (for Q2 2020), 604 (for Q3 2020), 606 (for Q4 2020), 608 (for Q1 2021), 610 (for Q2 2021), 612 (for Q3 2021), 614 (for Q4 2021) rendered within result screen 616 of user interface 200. Result screen 616 may also include a change/trend indicator for each of temporarily-spaced process efficacies (namely trend indicator 618, 620, 622, 624, 626, 628, 630, 632, respectively).

Additionally, such plurality of temporarily-spaced process efficacies (e.g., temporarily-spaced process efficacies 600, 602, 604, 606, 608, 610, 612, 614) may be displayed graphical in the form of time-based graph 634 for (in this example) user 36.

Online platform process 10 may determine 506 a long-term efficacy (e.g. long term efficacy 636) for the automated analysis process (e.g., automated analysis process 60) based, at least in part, upon the plurality of temporarily-spaced process efficacies (e.g., temporarily-spaced process efficacies 600, 602, 604, 606, 608, 610, 612, 614). In this particular example, the long-term efficacy (e.g. long term efficacy 636) for the automated analysis process (e.g., automated analysis process 60) is shown to be the percentage increase over the monitored period (e.g., Q1 2020 through Q4 2021). However, online platform process 10 may monitor many different things and express long term efficacy 636 many different ways.

For example and when determining 506 a long-term efficacy (e.g. long term efficacy 636) for the automated analysis process (e.g., automated analysis process 60) based, at least in part, upon the plurality of temporarily-spaced process efficacies 600, 602, 604, 606, 608, 610, 612, 614), online platform process 10 may monitor 508 the plurality of temporarily-spaced process efficacies 600, 602, 604, 606, 608, 610, 612, 614) to define an efficacy trend (e.g., upward, downward, stable) for the automated analysis process (e.g., automated analysis process 60 or automated analysis process 62).

Further and when determining 506 a long-term efficacy (e.g. long term efficacy 636) for the automated analysis process (e.g., automated analysis process 60) based, at least in part, upon the plurality of temporarily-spaced process efficacies 600, 602, 604, 606, 608, 610, 612, 614), online platform process 10 may confirm 510 that the efficacy trend is stable/trending upward (as shown in FIG. 7).

Additionally and when determining 506 a long-term efficacy (e.g. long term efficacy 636) for the automated analysis process (e.g., automated analysis process 60) based, at least in part, upon the plurality of temporarily-spaced process efficacies 600, 602, 604, 606, 608, 610, 612, 614), online platform process 10 may confirm 512 that the efficacy trend is not trending downward and, in the event of such a downward trend, user 36 (in this example) may be notified.

General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
iteratively processing a test truth set using an automated analysis process to generate a plurality of temporarily-spaced automated result sets;
iteratively determining a process efficacy for the automated analysis process based, at least in part, upon the test truth set and the plurality of temporarily-spaced automated result sets, thus defining a plurality of temporarily-spaced process efficacies; and
determining a long-term efficacy for the automated analysis process based, at least in part, upon the plurality of temporarily-spaced process efficacies.

2. The computer-implemented method of claim 1 further comprising:
defining the test truth set from a master truth set.

3. The computer-implemented method of claim 2 wherein defining the test truth set from a master truth set includes:
enabling a user to define narrowing criteria for the master truth set; and
applying the narrowing criteria to the master truth set to generate the test truth set, wherein the test truth set is a subset of the master truth set.

4. The computer-implemented method of claim 3 wherein the narrowing criteria concerns one or more of:
content type;
patient type; and
anomaly type.

5. The computer-implemented method of claim 1 wherein the test truth set includes a plurality of medical images and a plurality of related human-generated reports.

6. The computer-implemented method of claim 5 wherein the plurality of temporarily-spaced automated result sets includes a plurality of temporarily-spaced machine-generated reports.

7. The computer-implemented method of claim 6 wherein iteratively processing a test truth set using an automated analysis process to generate a plurality of temporarily-spaced automated result sets includes:
iteratively processing the plurality of medical images using the automated analysis process to generate the plurality of temporarily-spaced machine-generated reports included in the plurality of temporarily-spaced automated result sets, based upon the plurality of medical images.

8. The computer-implemented method of claim 6 wherein determining a long-term efficacy for the automated analysis process based, at least in part, upon the plurality of temporarily-spaced process efficacies includes:
monitoring the plurality of temporarily-spaced process efficacies to define an efficacy trend for the automated analysis process.

9. The computer-implemented method of claim 8 wherein determining a long-term efficacy for the automated analysis process based, at least in part, upon the plurality of temporarily-spaced process efficacies includes:
confirming that the efficacy trend is stable/trending upward.

10. The computer-implemented method of claim 8 wherein determining a long-term efficacy for the automated analysis process based, at least in part, upon the plurality of temporarily-spaced process efficacies includes:
confirming that the efficacy trend is not trending downward.

11. A computer program product, executed on a computing device, comprising:
iteratively processing a test truth set using an automated analysis process to generate a plurality of temporarily-spaced automated result sets;
iteratively determining a process efficacy for the automated analysis process based, at least in part, upon the test truth set and the plurality of temporarily-spaced automated result sets, thus defining a plurality of temporarily-spaced process efficacies; and
determining a long-term efficacy for the automated analysis process based, at least in part, upon the plurality of temporarily-spaced process efficacies.

12. The computer program product of claim 11 further comprising:
defining the test truth set from a master truth set.

13. The computer program product of claim 12 wherein defining the test truth set from a master truth set includes:
enabling a user to define narrowing criteria for the master truth set; and
applying the narrowing criteria to the master truth set to generate the test truth set, wherein the test truth set is a subset of the master truth set.

14. The computer program product of claim 13 wherein the narrowing criteria concerns one or more of:
content type;
patient type; and
anomaly type.

15. The computer program product of claim 11 wherein the test truth set includes a plurality of medical images and a plurality of related human-generated reports.

16. The computer program product of claim 15 wherein the plurality of temporarily-spaced automated result sets includes a plurality of temporarily-spaced machine-generated reports.

17. The computer program product of claim 16 wherein iteratively processing a test truth set using an automated analysis process to generate a plurality of temporarily-spaced automated result sets includes:
iteratively processing the plurality of medical images using the automated analysis process to generate the plurality of temporarily-spaced machine-generated reports included in the plurality of temporarily-spaced automated result sets, based upon the plurality of medical images.

18. The computer program product of claim 16 wherein determining a long-term efficacy for the automated analysis process based, at least in part, upon the plurality of temporarily-spaced process efficacies includes:
monitoring the plurality of temporarily-spaced process efficacies to define an efficacy trend for the automated analysis process.

19. The computer program product of claim 18 wherein determining a long-term efficacy for the automated analysis process based, at least in part, upon the plurality of temporarily-spaced process efficacies includes:
confirming that the efficacy trend is stable/trending upward.

20. The computer program product of claim 18 wherein determining a long-term efficacy for the automated analysis process based, at least in part, upon the plurality of temporarily-spaced process efficacies includes:
confirming that the efficacy trend is not trending downward.

21. A computing system, executed on a computing device, comprising:
iteratively processing a test truth set using an automated analysis process to generate a plurality of temporarily-spaced automated result sets;
iteratively determining a process efficacy for the automated analysis process based, at least in part, upon the test truth set and the plurality of temporarily-spaced automated result sets, thus defining a plurality of temporarily-spaced process efficacies; and
determining a long-term efficacy for the automated analysis process based, at least in part, upon the plurality of temporarily-spaced process efficacies.

22. The computing system of claim 21 further comprising:
defining the test truth set from a master truth set.

23. The computing system of claim 22 wherein defining the test truth set from a master truth set includes:
enabling a user to define narrowing criteria for the master truth set; and
applying the narrowing criteria to the master truth set to generate the test truth set, wherein the test truth set is a subset of the master truth set.

24. The computing system of claim 23 wherein the narrowing criteria concerns one or more of:
content type;
patient type; and
anomaly type.

25. The computing system of claim 21 wherein the test truth set includes a plurality of medical images and a plurality of related human-generated reports.

26. The computing system of claim 25 wherein the plurality of temporarily-spaced automated result sets includes a plurality of temporarily-spaced machine-generated reports.

27. The computing system of claim 26 wherein iteratively processing a test truth set using an automated analysis process to generate a plurality of temporarily-spaced automated result sets includes:
iteratively processing the plurality of medical images using the automated analysis process to generate the plurality of temporarily-spaced machine-generated reports included in the plurality of temporarily-spaced automated result sets, based upon the plurality of medical images.

28. The computing system of claim 26 wherein determining a long-term efficacy for the automated analysis process based, at least in part, upon the plurality of temporarily-spaced process efficacies includes:
monitoring the plurality of temporarily-spaced process efficacies to define an efficacy trend for the automated analysis process.

29. The computing system of claim 28 wherein determining a long-term efficacy for the automated analysis process based, at least in part, upon the plurality of temporarily-spaced process efficacies includes:
confirming that the efficacy trend is stable/trending upward.

30. The computing system of claim 28 wherein determining a long-term efficacy for the automated analysis process based, at least in part, upon the plurality of temporarily-spaced process efficacies includes:
confirming that the efficacy trend is not trending downward.
